(19) United States
(12) Patent Application Publication
(10) Pub. No.: US 2024/0245674 A1
(43) Pub. Date: Jul. 25, 2024

(54) DIAGNOSTIC METHODS AND COMPOSITIONS FOR TREATMENT OF CANCER

(71) Applicant: LINNAEUS THERAPEUTICS, INC., Haddonfield, NJ (US)

(72) Inventors: Christopher NATALE, Philadelphia, PA (US); Tina GARYANTES, Bradley Beach, NJ (US); Patrick MOONEY, Haddonfield, NJ (US)

(21) Appl. No.: 18/561,965

(22) PCT Filed: May 18, 2022
(86) PCT No.: PCT/US2022/029771

§ 371 Date: Nov. 17, 2023

Related U.S. Application Data
(60) Provisional application No. 63/190,484, filed on May 19, 2021.

(51) Int. Cl. A61K 31/473; A61K 39/395; A61P 35/00; G01N 33/574

(52) U.S. Cl. CPC A61K 31/473; A61K 39/3955; A61P 35/00; G01N 33/57484; G01N 2333/575; G01N 2333/726

(57) ABSTRACT

The present disclosure provides diagnostic methods and compositions usefid in the identification of patients and cancers that are amenable to treatment with cancer therapies, including agonist therapies against cancer targets that exist in normal and cancerous cells and tissues.

### CROSS REFERENCE TO RELATED APPLICATIONS

This Application claims the benefit of priority of U.S. Provisional Patent Application No. 63/190,484, filed May 19, 2021, which is incorporated herein by reference in its entirety.

### BACKGROUND OF THE DISCLOSURE

### Field of the Disclosure

The present disclosure relates to diagnostic methods and compositions useful in the treatment of neoplastic disorders including, e.g., cancer.

### Technical Background

Cancer has replaced cardiovascular disease as the number one cause of death in many developed nations and in many urban centers in the United States. Although recent advances in immune and targeted therapies have improved outcomes for many patients with advanced cancer, durable responses are achieved in only a minority of patients, treatment is frequently limited by significant side effects, and the repertoire of cancer targets is limited.

There is a significant unmet need for new cancer targets and cancer therapeutics, particularly small molecules that can be more cost effective than biologics. There is also a significant unmet need for methods of identifying and selecting patients who are likely to respond to a given anti-cancer treatment.

The present disclosure provides diagnostic methods and compositions useful in the identification of patients and cancers that are amenable to treatment with cancer therapies, including agonist therapies against cancer targets that exist in both normal and cancerous cells and tissues like G protein-coupled estrogen receptor 1 (GPER), a non-classical estrogen receptor. The disclosure also provides methods for treating disease states and conditions mediated through GPER receptors.

### SUMMARY OF THE DISCLOSURE

One aspect of the disclosure provides a method for identifying a patient whose cancer can respond to treatment with a cancer drug that binds to a cancer target in a target pathway, including obtaining first non-cancerous biological sample(s) from the patient before administering a test compound; administering an amount of test compound effective to produce a measurable change in one or more biomarkers in the target pathway; obtaining second non-cancerous biological sample(s) from the patient after administering the test compound; analyzing the second biological sample(s) for a change in the biomarker(s) after administration of the test compound as compared to the first sample(s); and identifying the patient as one whose cancer can respond to treatment with the cancer drug if the measurable change in one or more biomarkers in the target pathway corresponds to the measurable change in a healthy subject.

One aspect of the disclosure provides a method for identifying a cancer patient suitable for treatment with a cancer drug that binds to a cancer target in a target pathway, including obtaining first non-cancerous biological sample(s) from the patient before administering a test compound; administering an amount of test compound effective to produce a measurable change in one or more biomarkers in the target pathway; obtaining second biological sample(s) from the patient after administering the test compound; analyzing the second non-cancerous biological sample(s) for a change in the biomarker(s) after administration of the test compound as compared to the first sample(s); and identifying the cancer patient as suitable for treatment with the cancer drug if the measurable change in one or more biomarkers in the target pathway is substantially similar to the measurable change in a one or more cancer patients who responded to the cancer drug.

In some embodiments, the test compound comprises an agonist of the cancer target, an antagonist of the cancer target, or the cancer drug, and in some embodiments, the biomarker(s) is the cancer target of the cancer drug, and in some embodiments, the biomarker(s) is not the cancer target of the cancer drug.

One aspect of the disclosure provides a method for identifying a patient whose cancer can respond to treatment with LNS8801, including obtaining first biological sample(s) from the patient before administering a G protein-coupled estrogen receptor 1 (GPER) agonist; administering an amount of GPER agonist effective to produce a measurable change in one or more biomarkers of GPER activity in the patient; obtaining second biological sample(s) from the patient after administering the GPER agonist; analyzing the second sample(s) for a change in the biomarker(s) after administration of the GPER agonist as compared to the first sample(s); identifying the patient as one whose cancer can respond to treatment with LNS8801 if a measurable change in one or more biomarkers of GPER activity is measured.

One aspect of the disclosure provides a method for identifying a patient whose cancer can respond to treatment with LNS8801, including obtaining first biological sample(s) from the patient before administering a G protein-coupled estrogen receptor 1 (GPER) agonist; administering an amount of GPER agonist effective to produce a measurable change in biomarker(s) of GPER activity in a patient heterozygous or homozygous for wildtype GPER; obtaining second biological sample(s) from the patient after administering the GPER agonist; analyzing the second sample(s) for a change in the biomarker(s) after administration of the GPER agonist as compared to the first sample(s); identifying the patient as one whose cancer can respond to treatment with LNS8801 if a measurable change in biomarker(s) of GPER activity is measured.

One aspect of the disclosure provides a method for selecting a cancer patient suitable for treatment with LNS8801, including obtaining first biological sample(s) from the patient before administering a GPER agonist; administering an amount of GPER agonist effective to produce a measurable change in biomarker(s) of GPER activity in a patient; obtaining second biological sample(s) from the patient after administering the GPER agonist; analyzing the second sample(s) for a change in the biomarker(s) after administration of the GPER agonist as compared to the first sample(s); selecting the patient for treatment with LNS8801 if a measurable change in biomarker(s) of GPER activity is measured.

One aspect of the disclosure provides a method for identifying a patient whose cancer can respond to treatment with LNS8801, including: obtaining a biological sample; analyzing the sample to determine if the patient is heterozygous or homozygous for wildtype GPER; identifying the patient as one whose cancer can respond to treatment with LNS8801 if heterozygous or homozygous for GPER.

One aspect of the disclosure provides a method for selecting a cancer patient suitable for treatment with LNS8801, including obtaining a biological sample; analyzing the sample to determine if the patient is heterozygous or homozygous for wildtype GPER; selecting the patient for treatment with LNS8801 if heterozygous or homozygous for wildtype GPER.

One aspect of the disclosure provides a method for identifying a patient whose cancer will be refractive to treatment with LNS8801, including: obtaining a biological sample; analyzing the sample to determine if GPER is localized in the nucleus; identifying the patient as one whose cancer will be refractive to treatment with LNS8801 if GPER is localized in the nucleus.

One aspect of the disclosure provides a method for identifying a patient whose cancer can be refractive to treatment with LNS8801, including obtaining a biological sample; analyzing the sample to determine if the patient is heterozygous or homozygous for a GPER mutant; identifying the patient as one whose cancer can be refractive to treatment with LNS8801 if heterozygous or homozygous for a GPER mutant.

One aspect of the disclosure provides a method for selecting a cancer patient unsuitable for treatment with LNS8801, including obtaining a biological sample; analyzing the sample to determine if the patient is heterozygous or homozygous for a GPER mutant; selecting the patient as unsuitable for treatment with LNS8801 if heterozygous or homozygous for a GPER mutant.

In some embodiments, the GPER mutant comprises a P16L mutation.

One aspect of the disclosure provides a method for identifying a patient whose cancer can respond to treatment with LNS8801, including obtaining first biological sample(s) from the patient before administering LNS8801; administering an amount of LNS8801 effective to produce a measurable increase in prolactin in a patient; obtaining second biological sample(s) from the patient after administering the LNS8801; analyzing the second sample(s) for an increase in prolactin of greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, greater than about 100% over the first biological sample(s) after administration of the LNS8801; identifying the patient as one whose cancer can respond to treatment with LNS8801 if a greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 100% increase in prolactin is measured.

One aspect of the disclosure provides a method for selecting a cancer patient suitable for treatment with LNS8801, including obtaining first biological sample(s) from the patient before administering LNS8801; administering an amount of LNS8801 effective to produce a measurable increase in prolactin in a patient; obtaining second biological sample(s) from the patient after administering the LNS8801; analyzing the sample(s) for an increase in prolactin of greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 100% over the first sample(s) after administration of the LNS8801; selecting the patient for treatment with LNS8801 if a greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 100% increase in prolactin is measured.

In some embodiments, the prolactin increase is calculated by dividing the average serum prolactin concentration at about 4, about 7 and about 10 hours after LNS8801 administration by the average prolactin concentration pre-dose, and about 0.5, about 1 and about 2 hours after administration.

One aspect of the disclosure provides a method of treating cancer in a patient in need thereof, including obtaining a biological sample from the patient; determining if the patient is heterozygous or homozygous for wildtype GPER from the sample; determining the patient is amenable to treatment with LNS8801 if heterozygous or homozygous for wildtype GPER; and administering to the patient an effective amount of LNS8801.

One aspect of the disclosure provides a method of treating cancer in a patient in need thereof, including obtaining first biological sample(s) from the patient before administering a GPER agonist; administering an amount of G protein-coupled estrogen receptor 1 (GPER) agonist effective to produce a measurable change in one or more biomarkers of GPER activity in a patient heterozygous or homozygous for wildtype GPER; obtaining second biological sample(s) from the patient at one or more times after administering the GPER agonist; analyzing the sample(s) for a change in the biomarker(s) after administration of the GPER agonist; determining the patient is amenable to treatment with LNS8801 if a measurable change in one or more biomarkers of GPER activity is measured; and administering to the patient an effective amount of LNS8801.

In some embodiments, the GPER agonist includes 2-methoxyestradiol, aldosterone, estradiol, ethynylestradiol, LNS8801, G-1, genistein, hydroxytyrosol, niacin, nicotinamide, quercetin, and resveratrol, and in some embodiments the GPER agonist is LNS8801.

In some embodiments of the various aspects of the disclosure, the patient is homozygous for wildtype GPER, and in some embodiments, the patient is homozygous for a GPER mutant.

In some embodiments of the various aspects of the disclosure, the test compound, the GPER agonist, and the LNS8801 are administered in one dose or in two or more doses, and the effective amount of test compound can be a clinical dose, a sub-clinical dose, or a microdose. In some embodiments, the sub-clinical dose includes between about 1.1% and 99.9 percent of the clinical dose, and in some embodiments, the microdose comprises between about 0.01% and 1% of the clinical dose.

In some embodiments of the various aspects of the disclosure, the biological sample(s) include one or more cells and/or tissues that are not cancerous, and in some embodiments, the biological sample(s) include cells and/or tissues that are all non-cancerous. In some embodiments, the first biological sample(s) are obtained not more than 30 days prior to administering the test compound, the GPER agonist, or the LNS8801, and in some embodiments, the first biological sample(s) is collected at the same time of day as the second biological sample(s). In some embodiments, the biomarker(s) of the test compound, the GPER agonist, and the LNS8801 activity include one or more molecular biomarkers, imaging biomarkers or non-invasively measurable biomarkers, which biomarkers include, in embodiments, circulating biomarker(s) and/or systemic biomarker(s), and/or biomarker(s) that are localized to the first and/or second biological sample(s).

In some embodiments that include administering an effective amount of GPER agonist or LNS8801, the biomarker(s) comprise circulating biomarker(s), which include a change in prolactin level, insulin level, c-Myc and/or glucose level, which change, in embodiments, includes an increase in prolactin level or activity, an increase in insulin level or activity or a decrease in c-Myc level or activity. In some embodiments, the biomarker of GPER agonist activity (including LNS88801) is an increase in circulating prolactin level.

In some embodiments wherein the biomarker of GPER agonist activity (including LNS88801) is an increase in circulating prolactin level, the prolactin exhibits an about 1.25-fold induction, an about 1.30-fold induction, an about 1.35-fold induction, an about 1.40-fold induction, an about 1.45-fold induction, an about 1.50-fold induction, an about 1.55-fold induction, an about 1.60-fold induction, an about 1.65-fold induction, an about 1.70-fold induction, an about 1.75-fold induction, an about 1.80-fold induction, an about 1.85-fold induction, an about 1.90-fold induction, an about 1.95-fold induction, an about 2.0-fold induction after administration of an effective amount of GPER agonist, including LNS8801.

In some embodiments wherein the biomarker of GPER agonist activity (including LNS88801) is an increase in circulating prolactin level, the prolactin increases above a threshold at an average of about 4 hours (+/−20 min), about 7 hours (+/−45 min) and about 12 hours (+/−2 hours) divided by the average concentration of prolactin at pre-dose and 30 min, 1 hour and 2 hours post-dose, and the increase is more than 25% to monotherapy or more than 40% to monotherapy and less to combination therapy with a PD-1 inhibitor.

Some embodiments of the aspects of the disclosure providing methods of treating cancer further include concurrently, coincidently or sequentially administering a PD-1 inhibitor including one or more of pembrolizumab, nivolumab, cemiplimab, JTX-4014, spartalizumab, camrelizumab, sintilimab, tislelizumab, toripalimab, dostarlimab, INCMGA00012 (MGA012), AMP-224, and AMP-514. In some embodiments, the PD-1 inhibitor includes pembrolizumab.

### BRIEF DESCRIPTION OF THE DRAWINGS

FIG. 1 shows the pharmacokinetics of LNS8801 on Day 1 (FIG. 1A) and Day 3 (FIG. 1B) from the 10, 40, and 125 mg capsule cohorts.

FIG. 2(A) shows representative pre- and on treatment images of c-Myc immunohistochemistry on uveal melanoma patient biopsies. FIG. 2(B) shows percent change of c-Myc immunohistochemistry scoring on pre- and on-treatment biopsies.

FIG. 3 show prolactin induction versus best RECIST response after LNS8801 monotherapy.

FIG. 4 shows the best RECIST response in prolactin-responders vs non-prolactin-responders. Statistical significance determined using Mann-Whitney test.

FIG. 5 shows a schematic representing a GPER-related biochemical pathway with activities in certain cancers.

### DETAILED DESCRIPTION

The present inventors have unexpectedly determined that that the nonclassical estrogen receptor, GPER, is a therapeutic target for cancers not classically known to be sex steroid responsive, including melanoma. Further, it was determined that GPER agonists, as opposed to the standard antagonist anti-cancer therapies that work by inhibiting receptors of activated oncogenes with the goal of killing cancer cells, activate GPER to induce differentiation in certain cell types that is associated with (1) increased expression of differentiation antigens that are recognized by cytotoxic T cells and (2) increased expression of HLA class I proteins. Together, these effects render tumor cells more antigenic and vulnerable to killing by immune cells. Unexpectedly, measuring a systemic response (e.g., not in the cancer) to a GPER agonist is a reliable predictor of the response of a patient's cancer to the agonist. Measuring responses in the tumor, such as c-myc expression, are also predictive of patient response.

### Definitions

As used herein, the terms below have the meanings indicated.

As used herein and in the appended claims, the singular forms “a,” “an,” and “the” include plural reference unless the context clearly dictates otherwise.

As used herein, the term “about” means plus or minus 20% of the numerical value of the number with which it is being used. Therefore, about 50% means in the range of 40%-60%.

As used herein, the terms “comprising” and “including” are used synonymously and indicate one or more recited elements may include other elements not specifically recited. For example, a composition that “comprises” or “includes” a polypeptide sequence may contain the sequence alone or in combination with other sequences or ingredients.

As used herein, the term “consists of” or “consisting of” means that the compound, composition, formulation or the method includes only the elements, steps, or ingredients specifically recited in the particular claimed embodiment or claim.

As used herein, the term “consisting essentially of” or “consists essentially of” means that the compound, composition, formulation or the method includes only the elements, steps or ingredients specifically recited in the particular claimed embodiment or claim and may optionally include additional elements, steps or ingredients that do not materially affect the basic and novel characteristics of the particular embodiment or claim. For example, the only active ingredient(s) in the formulation or method that treats the specified condition (e.g., cancer and/or obesity) is the specifically recited therapeutic(s) in the particular embodiment or claim.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used herein, the term “patient”, “subject” and “individual” are interchangeable and may be taken to mean any living organism, which may be treated with compounds of the present invention. As such, the terms “patient” and “subject” may include, but is not limited to, any non-human mammal, primate or human. In some embodiments, the “patient” or “subject” is an adult, child, infant, or fetus. In some embodiments, the “patient” or “subject” is a human. In some embodiments, the “patient” or “subject” is a mammal, such as mice, rats, other rodents, rabbits, dogs, cats, swine, cattle, sheep, horses, primates, or humans.

The term “biological sample”, “sample,” and “test sample”, as used herein, refer to a composition that is obtained or derived from a patient or subject of interest that contains a cellular and/or other molecular entity that is to be characterized and/or identified, for example based on physical, biochemical, chemical and/or physiological characteristics. In one embodiment, the definition encompasses blood and other liquid samples of biological origin and tissue samples such as a biopsy specimen or tissue cultures or cells derived therefrom. The source of the tissue sample may be solid tissue as from a fresh, frozen and/or preserved organ or tissue sample or biopsy or aspirate; blood or any blood constituents; bodily fluids; and cells from any time in gestation or development of the subject or plasma.

The terms “biological sample”, “sample,” and “test sample” include samples that have been manipulated in any way after their procurement, such as by treatment with reagents, solubilization, or enrichment for certain components, such as proteins or polynucleotides, or embedding in a semi-solid or solid matrix for sectioning purposes. For the purposes herein a “section” of a tissue sample is meant a single part or piece of a tissue sample, e.g., a thin slice of tissue or cells cut from a tissue sample. Samples include, but not limited to, primary or cultured cells or cell lines, or bodily fluid, where “bodily fluid” can be any useful fluid, including without limitation one or more of peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, umbilical cord blood, tissue culture medium, tissue extracts such as homogenized tissue, tumor tissue, cellular extracts, and combinations thereof. In some embodiments, the bodily fluid comprises blood, serum.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., lung etc.), the size and type of the tumor, among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue from within the tumor. A diagnosis or prognosis made by endoscopy or radiographic guidance can require a “core-needle biopsy”, or a “fine-needle aspiration biopsy” which generally obtains a suspension of cells from within a target tissue. Biopsy techniques are discussed, for example, in Harrison's Principles of Internal Medicine, Kasper, et al., eds., 16th ed., 2005, Chapter 70, and throughout Part V.

In some embodiments, the sample is used in a diagnostic assay. In some embodiments, the sample comprises normal, wild type cells and/or tissue. That is, the sample is free from cells that are cancerous or exhibit cancer-like characteristics (a “non-cancerous biological sample”), where “cancer-like characteristics” include one or more biomarkers that exhibit activity or abundance more common to a cancer than to a non-cancerous cells or tissue, to frank cancer, in which the cells or tissues exhibit one or more of characteristics such as uncontrolled proliferation, immortality, metastatic potential, rapid growth and proliferation rate, and certain characteristic morphological features. In some embodiments, the non-cancerous biological sample includes a germline cell, a somatic cell, or combinations thereof.

In some embodiments, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood.

In some embodiments, a “test compound” is administered that may “specifically or selectively bind” to a “cancer target” and cause a “functional effect” or “measurable change” in the cancer target or biomarker downstream of the cancer target in a “target pathway”, which terms are all defined elsewhere herein. The test compound(s) can be, for example, (anti-)cancer drugs, GPER agonists, or LNS8801.

In some embodiments, a sample(s) is obtained prior to administration of a test compound or a course of therapy with, e.g., (anti-)cancer drug(s), GPER agonist(s), or LNS8801. Samples taken before administration of a test compound or course of therapy can serve as a “reference sample”. More generally, a “reference sample,” refers to any sample, standard, or level that is used for comparison purposes. In one embodiment, a reference sample is obtained from a healthy and/or non-diseased part of the body (e.g., tissue or cells) of the same subject or patient. In another embodiment, a reference sample is obtained from an untreated tissue and/or cell of the body of the same subject or patient. In yet another embodiment, a reference sample is obtained from a healthy and/or non-diseased part of the body (e.g., tissues or cells) of an individual who is not the subject or patient. In even another embodiment, a reference sample is obtained from an untreated tissue and/or cell part of the body of an individual who is not the subject or patient.

In certain embodiments, a reference sample is a single sample or a combination of multiple samples from the same subject or patient that are obtained at one or more different time points than when the test sample is obtained. For example, a reference sample is obtained at an earlier time point from the same subject or patient than when the test sample is obtained. Such reference sample may be useful if the reference sample is obtained during initial diagnosis of cancer and the test sample is later obtained after one or more administrations of a course of therapy has been administered.

In certain embodiments, a reference sample includes all types of samples as defined above under the term “biological sample” that is obtained from one or more individuals who is not the subject or patient. In certain embodiments, a reference sample is obtained from one or more individuals with or without a neoplastic disorder (e.g., cancer) who are not the subject or patient.

In certain embodiments, a reference sample is a combination of multiple samples from one or more healthy individuals who are not the subject or patient. In certain embodiments, a reference sample is a combination of multiple samples from one or more individuals with a disease or disorder (e.g., an angiogenic disorder such as, for example, cancer) who are not the subject or patient. In certain embodiments, a reference sample is pooled RNA samples from normal tissues or pooled plasma or serum samples from one or more individuals who are not the subject or patient. In certain embodiments, a reference sample is pooled RNA samples from tumor tissues or pooled plasma or serum samples from one or more individuals with a disease or disorder (e.g., an angiogenic disorder such as, for example, cancer) who are not the subject or patient.

In embodiments, a sample(s) is obtained from a subject or patient after at least one administration of a test compound or at least one treatment with a GPER agonist, LNS8801 or cancer therapy. In some embodiments, a sample is obtained from a patient before cancer has metastasized. In certain embodiments, a sample is obtained from a patient after cancer has metastasized.

The term “marker” or “biomarker” refers to a molecule (typically protein, nucleic acid, carbohydrate, or lipid) that is present in the cell (e.g., gene sequence containing one or more mutations), expressed in the cell, expressed on the surface of a cancer cell or secreted by a cancer cell in comparison to a non-cancer cell, and which is useful for the diagnosis of cancer, for providing a prognosis, and for preferential targeting of a pharmacological agent to the cancer cell. Such markers are often molecules that are overexpressed in a cancer cell in comparison to a non-cancer cell, for instance, 2-fold overexpression, 3-fold overexpression, 10-fold overexpression or more in comparison to a normal cell. Further, a marker can be a molecule that is inappropriately synthesized in the cancer cell, for instance, a molecule that contains deletions, additions (including amplifications/multiple copies) or mutations in comparison to the molecule expressed on a normal cell. Alternatively, such biomarkers are molecules that are underexpressed in a cancer cell in comparison to a non-cancer cell, for instance, 2-fold underexpression, 3-fold underexpression, 10-fold underexpression, or more. Such differences in expression and/or regulation between normal and cancerous or pre-cancerous cells can also be referred to as “differentially expressed” or “differentially regulated”. Further, a marker can be a molecule that is inappropriately synthesized in cancer, for instance, a molecule that contains deletions, additions or mutations in comparison to the molecule expressed in a normal cell.

The term “molecular biomarker” refers to refer to non-imaging biomarkers that have biophysical properties, which allow their measurements in biological samples (e.g., plasma, serum, cerebrospinal fluid, bronchoalveolar lavage, biopsy, urine) and include nucleic acids-based biomarkers such as gene mutations or polymorphisms and quantitative gene expression analysis, and the presence, abundance and/or activity of peptides, proteins, lipids metabolites, and other small molecules.

The term “imaging biomarker” refers to biomarkers that are detectable in an image, e.g., x-ray, computerized tomography (CT) or magnetic resonance imaging (MRI).

The term “non-invasively measurable biomarkers” refers to non-imaging biomarkers that do not require invasive collection techniques like venipuncture for collection of a blood sample. Examples include, e.g., blood pressure, flushing, breath and the like.

It will be understood by the skilled artisan that biomarkers may be used in combination with other biomarkers or tests for any of the uses, e.g., prediction, diagnosis, or prognosis of cancer, amenability of a cancer to specific treatment and the like, disclosed herein.

Expression levels/amount of biomarkers, and, e.g., mutations in biomarkers can be determined qualitatively and/or quantitatively based on any suitable criterion known in the art, including but not limited to mRNA, cDNA, proteins, and protein fragments. A “measurable change” in biomarker refers to a difference in a biomarker that is qualitatively and/or quantitatively measurable between and among samples. A measurable change in a biomarker may result from the administration of a test compound, GPER agonist, LNS8801, a cancer drug/therapy. A measurable change may also result from the presence in one or more pre-cancerous or cancerous cells in the sample.

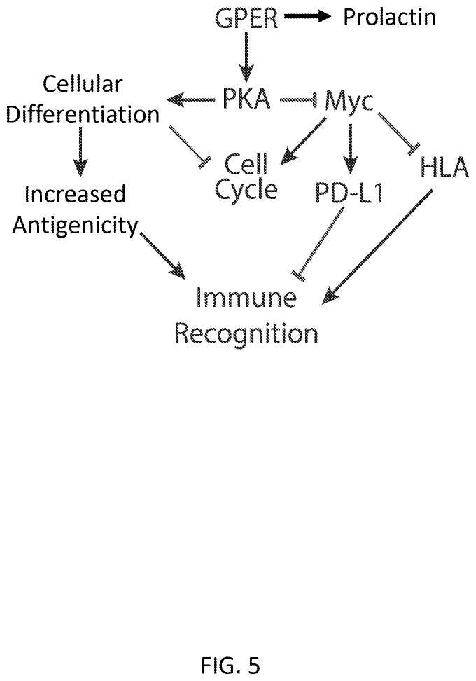

The term “target pathway” refers to a biochemical pathway that is known to be (or hypothesized to be) involved in the process of cellular transformation of normal cells into cancer cells or propagation of cancer cells. For example, angiogenesis is stimulated in many cancers through the VEGF/VEGR receptor pathway. VEGF binds VEGF receptor, which, in turn, works through P13K and Akt/PKB to enhance endothelial cell survival and vascular permeability. VEGF and VEGF receptor also work through PKC, SPK, Ras, Raf, MEK and ERK to promote endothelial cell proliferation. In concert, angiogenesis is increased, feeding cancer cells. Another example is shown in FIG. 5. GPER upregulation stimulates PKA, which downregulates c-Myc. c-Myc downregulation results in upregulation (a reduction of inhibition) of HLA and downregulation (a reduction of upregulation) of PD-LI and cell cycle stimulation. The result of these activities is increased immune recognition of certain cancerous cells.

In the exemplary pathways, potentially any of the described proteins (or genes, mRNAs, etc., associated therewith) may be a suitable “cancer target”. Cancer targets can be molecules whose function and/or abundance is altered through the addition of“test compound”, e.g., cancer target agonist or antagonist where the cancer target is a protein. Cancer targets can also be selected and used according to embodiments of the disclosure without the use of a test compound. Cancer targets can serve as biomarkers, as well as downstream effects of the cancer target. Regarding FIG. 5, agonism of GPER results in, e.g., upregulation of prolactin and downregulation (by enhanced degradation) of c-Myc. In this instance, the GPER pathway is a “target pathway”, GPER can serve as a “cancer target”, and prolactin or c-Myc can serve as biomarkers.

As used herein, “RECIST” refers to Response Evaluation Criteria In Solid Tumors and is a set of published rules (from by an international collaboration including the European Organisation for Research and Treatment of Cancer (EORTC), National Cancer Institute of the United States, and the National Cancer Institute of Canada Clinical Trials Group) that define when tumors in cancer patients improve (“respond”), stay the same (“stabilize”), or worsen (“progress”) during treatment. RECIST provides tumor-centric (as opposed to patient-centric) evaluation criteria that include (1) baseline documentation of “target” and “non-target” lesions. (2) evaluation of response by target regions (e.g., complete response (CR), partial response (PR), stable disease (SD) and progressive disease (PD)), non-target regions, and evaluation of the best overall response recorded from the start of the treatment until disease progression/recurrence.

The phrase “specifically (or selectively) binds” when referring to a protein, nucleic acid, antibody, or small molecule compound refers to a binding reaction that is determinative of the presence of the protein or nucleic acid, e.g., the binding of LNS8801 to GPER.

The phrase “functional effects” in the context of assays for testing compounds that modulate a cancer target includes the determination of a parameter that is indirectly or directly under the influence of cancer target. A functional effect includes ligand binding activity, transcriptional activation or repression, the ability of cells to proliferate, the ability to migrate, among others. “Functional effects” include in vitro, in vivo, and ex vivo activities.

By “determining the functional effect” is meant assaying for a compound that increases or decreases a parameter that is indirectly or directly under the influence of a cancer target of the disclosure, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface markers. The functional effects can be evaluated by many means known to those skilled in the art, e.g., microscopy for quantitative or qualitative measures of alterations in morphological features, measurement of changes in RNA or protein levels, measurement of RNA stability, identification of downstream or reporter gene expression (CAT, luciferase, p-gal, GFP and the like), e.g., via chemiluminescence, fluorescence, colorimetric reactions, antibody binding, inducible markers, etc.

The terms “protein”, “polypeptide” and “peptide” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

In certain embodiments, by “correlate” or “correlating” is meant comparing, in any way, the performance and/or results of a first test, analysis or protocol with the performance and/or results of a second test, analysis or protocol. For example, one may use the results of a first test, analysis or protocol in carrying out a second test or protocol and/or one may use the results of a first test, analysis or protocol to determine whether a second test or analysis or protocol should be performed. With respect to an embodiment of gene expression or protein function test, analysis or protocol, one may use the results of the gene expression or protein function test, analysis or protocol to determine whether a specific therapeutic regimen should be performed.

A “disorder” is any condition that would benefit from treatment including, but not limited to, chronic and acute disorders or diseases including those pathological conditions which predispose the mammal to the disorder in question. Disorders include neoplastic disorders. “Neoplastic disorder” as used herein refers to any condition involving abnormal cellular growth. Non-limiting examples of angiogenic disorders to be treated herein include malignant and benign tumors; leukemias and lymphoid malignancies; and tumor (cancer) metastasis.

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells. Of interest in the disclosure are “cancers” whose development, progression, and or response to therapy, may be influenced by endogenous, and/or pharmacologic activation of GPER signaling (including the prevention of cancer, prevention of the reoccurrence of cancer, and the inhibition of the progression of cancer), including melanoma, pancreatic, lymphomas, uveal melanoma, non-small cell lung cancer, breast, reproductive and other hormone-dependent cancers, leukemia, colon cancer, prostate, and bladder cancer.

“Abnormal cell growth”, as used herein, unless otherwise indicated, refers to cell growth that is independent of normal regulatory mechanisms (e.g., loss of contact inhibition).

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Inhibitors,” “activators,” and “modulators” of the markers are used to refer to activating, inhibiting (inhibitory), or modulating molecules identified using in vitro and in vivo assays of cancer targets and biomarkers. Inhibitors are compounds that, e.g., bind to, partially or totally block activity, decrease, prevent, delay activation, inactivate, desensitize, or down regulate the activity or expression of cancer targets and biomarkers. “Activators” are compounds that increase, open, activate, facilitate, enhance activation, sensitize, agonize, or up regulate activity of cancer targets and biomarkers, e.g., agonists. Inhibitors, activators, or modulators also include genetically modified versions of cancer targets and biomarkers, e.g., versions with altered activity, as well as naturally occurring and synthetic ligands, antagonists, agonists, antibodies, peptides, cyclic peptides, nucleic acids, antisense molecules, ribozymes, RNAi and siRNA molecules, small organic molecules and the like. Such assays for inhibitors and activators include, e.g., expressing cancer targets and/or biomarkers in vitro, in cells, or cell extracts, applying putative modulator compounds, and then determining the functional effects on activity, as described above.

The term “genotype” as used herein means the nucleotide characters at a particular nucleotide variant marker (or locus) in either one allele or both alleles of a gene (or a particular chromosome region). With respect to a particular nucleotide position of a gene of interest, the nucleotide(s) at that locus or equivalent thereof in one or both alleles form the genotype of the gene at that locus. A genotype can be homozygous or heterozygous, e.g., two wild-type copies of the GPER gene (homozygous wild-type), one wild-type GPER allele and one mutant allele (heterozygous) or two GPER mutant alleles (homozygous mutant). Accordingly, “genotyping” means determining the genotype, that is, the nucleotide(s) at a particular gene locus. Genotyping can also be done by determining the amino acid variant at a particular position of a protein which can be used to deduce the corresponding nucleotide variant(s).

“Mutation” is defined herein as a specific change at a genomic location, i.e.: Chromosome, start, stop, reference base, alternate base, variant type (SNP, INS, DEL) etc. The altered genetic location (gene) or mRNA or protein product of a mutation can be referred to as a “mutant”.

“Detection,” “detectable” and grammatical equivalents thereof refer to ways of determining the presence and/or quantity and/or identity of a target nucleic acid sequence (e.g., gene, mRNA) or protein sequence resulting therefrom. In some embodiments, detection occurs by amplifying the target nucleic acid sequence. In other embodiments, sequencing of the target nucleic acid can be characterized as “detecting” the target nucleic acid. A label attached to the probe can include any of a variety of different labels known in the art that can be detected by, for example, chemical or physical means. Labels that can be attached to probes may include, for example, fluorescent and luminescence materials. In some embodiments, detection occurs by protein activity assessment (measurement of protein activity, either direct or indirect) or protein separation techniques (e.g., isoelectric focusing, chromatographic techniques, electrophoretic techniques), Western blotting or protein identification (e.g., de novo peptide sequencing, peptide mass fingerprinting).

The terms “effective amount”, “amount effective to”, and the like, are used herein interchangeably and may refer to the amount of an active agent or pharmaceutical compound or composition or test compound that elicits a measurable clinical, biological or medicinal change or response in a biomarker, cell, tissue, system, or patient.

A “pharmaceutically effective amount” is used to describe a measurable clinical, biological or medical response may include, for example, one or more of the following: (1) preventing a disease, condition or disorder in an individual that may be predisposed to the disease, condition or disorder but does not yet experience or display pathology or symptoms of the disease, condition or disorder, (2) inhibiting a disease, condition or disorder in an individual that is experiencing or displaying the pathology or symptoms of the disease, condition or disorder or arresting further development of the pathology and/or symptoms of the disease, condition or disorder, and (3) ameliorating a disease, condition or disorder in an individual that is experiencing or exhibiting the pathology or symptoms of the disease, condition or disorder or reversing the pathology and/or symptoms experienced or exhibited by the individual.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount. A prophylactically effective amount encompasses an amount sufficient to confer benefit, e.g., clinical benefit.

In the case of pre-cancerous, benign, early or late-stage tumors, the therapeutically effective amount of the cancer drug, GPER agonist, or LNS8801 may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit or delay, to some extent, tumor growth or tumor progression; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The term “combination therapy” means the administration of two or more therapeutic agents to treat a medical condition or disorder described in the present disclosure. Such administration encompasses co-administration of these therapeutic agents in a substantially simultaneous manner, such as in a single capsule, or dosage presentation, having a fixed ratio of active ingredients or in multiple, separate capsules for each active ingredient. For example, administration of LNS8801 and a PD-1 inhibitor is a combination therapy according to the disclosure and claims. In addition, such administration also encompasses use of each type of therapeutic agent in a sequential manner in the same patient, with delivery of the individual therapeutics separated by 1-24 hours, 1-7 days, or 1 or more weeks. In either case, the treatment regimen will provide beneficial effects of the drug combination in treating the conditions or disorders described herein.

The terms “administer,” “administering” or “administration” as used herein refer to either directly administering a compound or pharmaceutically acceptable salt of the compound or a composition to a subject.

The term “treating” may be taken to mean prophylaxis of a specific disorder, disease or condition, alleviation of the symptoms associated with a specific disorder, disease or condition and/or prevention of the symptoms associated with a specific disorder, disease or condition. In some embodiments, the term refers to slowing the progression of the disorder, disease or condition or alleviating the symptoms associated with the specific disorder, disease or condition. In some embodiments, the term refers to alleviating the symptoms associated with the specific disorder, disease or condition. In some embodiments, the term refers to alleviating the symptoms associated with the specific disorder, disease or condition. In some embodiments, the term refers to restoring function which was impaired or lost due to a specific disorder, disorder or condition.

The term “preventing” may be taken to mean to prevent a specific disorder, disease or condition and/or prevent the reoccurrence of a specific disorder, disease or condition.

It is to be understood that this invention is not limited to the particular processes, formulations, compound, compositions, or methodologies described, as these may vary. It is also to be understood that the terminology used in the description is for the purpose of describing the particular versions or embodiments only and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F.

M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. 1. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction,(Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### Methods

Generally, provided herein are methods for selecting, identifying and treating patients and cancers that can respond to cancer drugs/therapies generally, GPER agonists generally, but also LNS8801 (“1-((3aS,4R,9bR)-4-(6-bromobenzo[d][1,3]dioxol-5-yl)-3a,4,5,9b-tetrahydro-3H-cyclopenta[c]quinolin-8-yl)ethan-1-one” and “SRR G-1”) specifically.

In one aspect, the disclosure provides a method for identifying a patient whose cancer can respond to treatment with a cancer drug that binds to a cancer target in a target pathway, comprising obtaining first non-cancerous biological sample(s) from the patient before administering a test compound; administering an amount of test compound effective to produce a measurable change in one or more biomarkers in the target pathway; obtaining second non-cancerous biological sample(s) from the patient after administering the test compound; analyzing the second biological sample(s) for a change in the biomarker(s) after administration of the test compound as compared to the first sample(s); and identifying the patient as one whose cancer can respond to treatment with the cancer drug if the measurable change in one or more biomarkers in the target pathway corresponds to the measurable change in a healthy subject.

One aspect of the disclosure provides a method for identifying a cancer patient suitable for treatment with a cancer drug that binds to a cancer target in a target pathway, comprising obtaining first non-cancerous biological sample(s) from the patient before administering a test compound; administering an amount of test compound effective to produce a measurable change in one or more biomarkers in the target pathway; obtaining second biological sample(s) from the patient after administering the test compound; analyzing the second non-cancerous biological sample(s) for a change in the biomarker(s) after administration of the test compound as compared to the first sample(s); and identifying the cancer patient as suitable for treatment with the cancer drug if the measurable change in one or more biomarkers in the target pathway is substantially similar to the measurable change in a one or more cancer patients who responded to the cancer drug.

In embodiments, biological sample(s) can be non-cancerous, i.e., containing a negligible number (e.g., <5%, <2%, <1%) of cancerous cells (or cell products, and the like) or not containing any cancerous cells or cell products. Such biological samples are intended to reflect the response of a “normal” sample, which, in embodiments, provides insight regarding how a cancer can respond to the test compound. GPER provides an illustrative example. GPER signaling in normal host tissue has correlated with an anti-cancer effect in tumors. That is, both normal and cancerous tissues respond in concert, both responding or not. Thus, germline variations likely underlie this association. When a test compound binds to a cancer target in a target pathway producing a measurable change in one or more biomarkers, which cancer target and target pathway also exist in normal cells, less invasive, easily accessible samples (e.g., saliva, cheek cells and the like) may be used to identifying a cancer patient who is suitable or a patient whose cancer can respond to treatment with a cancer drug that binds to a cancer target in a target pathway.

In some embodiments, the test compound comprises an agonist of the cancer target, or an antagonist of the cancer target, or the cancer drug. In some embodiments, the biomarker(s) is the cancer target of the cancer drug, and in embodiments, the biomarker(s) is not the cancer target of the cancer drug.

One aspect of the disclosure provides a method for identifying a patient whose cancer can respond to treatment with LNS8801, including obtaining first biological sample(s) from the patient before administering a GPER agonist; administering an amount of GPER agonist effective to produce a measurable change in one or more biomarkers of GPER activity in the patient; obtaining second biological sample(s) from the patient after administering the GPER agonist; analyzing the second sample(s) for a change in the biomarker(s) after administration of the GPER agonist as compared to the first sample(s); identifying the patient as one whose cancer can respond to treatment with LNS8801 if a measurable change in one or more biomarkers of GPER activity is measured.

In some embodiments, the amount of GPER agonist effective to produce a measurable change in one of more biomarkers of GPER activity in the patient is an amount known to be effective in producing a measurable change in a patient heterozygous or homozygous for wildtype GPER, i.e., a reference sample. The reference sample, as described above, can be from the patient (e.g., with prior knowledge of GPER allelic status from a prior treatment) or from one or more people who (1) are not the patient and (2) have a known allelic makeup (heterozygous or homozygous for wild-type GPER) and (3) have known prior dosing information that was observed to produce the desired effect.

One aspect of the disclosure provides a method for selecting a cancer patient suitable for treatment with LNS8801, including obtaining first biological sample(s) from the patient before administering a GPER agonist; administering an amount of GPER agonist effective to produce a measurable change in biomarker(s) of GPER activity in a patient; obtaining second biological sample(s) from the patient after administering the GPER agonist; analyzing the second sample(s) for a change in the biomarker(s) after administration of the GPER agonist as compared to the first sample(s); selecting the patient for treatment with LNS8801 if a measurable change in biomarker(s) of GPER activity is measured.

In some embodiments, determining whether a patient exhibits a normal response to GPER agonism can be performed with a GPER agonist other than LNS8801. In some embodiments, the GPER agonist can be, e.g., 2-methoxyestradiol, aldosterone, estradiol, ethynylestradiol, G-1, genistein, hydroxytyrosol, niacin, nicotinamide, quercetin, or resveratrol. If the response of one or more biomarkers demonstrate a functioning GPER pathway, the patient can be selected for treatment with LNS8801.

One aspect of the disclosure provides a method for identifying a patient whose cancer can respond to treatment with LNS8801, including: obtaining a biological sample; analyzing the sample to determine if the patient is heterozygous or homozygous for wild-type GPER; identifying the patient as one whose cancer can respond to treatment with LNS8801 if heterozygous or homozygous for GPER.

One aspect of the disclosure provides a method for selecting a cancer patient suitable for treatment with LNS8801, including obtaining a biological sample; analyzing the sample to determine if the patient is heterozygous or homozygous for wildtype GPER: selecting the patient for treatment with LNS8801 if heterozygous or homozygous for wildtype GPER.

One aspect of the disclosure provides a method for identifying a patient whose cancer will be refractive to treatment with LNS8801, including: obtaining a biological sample; analyzing the sample to determine if GPER is localized in the nucleus; identifying the patient as one whose cancer will be refractive to treatment with LNS8801 if GPER is localized in the nucleus.

One aspect of the disclosure provides a method for identifying a patient whose cancer can be refractive to treatment with LNS8801, including obtaining a biological sample; analyzing the sample to determine if the patient is heterozygous or homozygous for a GPER mutant; identifying the patient as one whose cancer can be refractive to treatment with LNS8801 if heterozygous or homozygous for a GPER mutant.

One aspect of the disclosure provides a method for selecting a cancer patient unsuitable for treatment with LNS8801, including obtaining a biological sample; analyzing the sample to determine if the patient is heterozygous or homozygous for a GPER mutant; selecting the patient as unsuitable for treatment with LNS8801 if heterozygous or homozygous for a GPER mutant.

In some embodiments, refractivity can be complete or partial. Patients or cancers that are homozygous for mutant GPER are likely to be completely refractive to LNS8801 treatment; whereas, patients or cancers that are heterozygous for wild-type GPER may exhibit partial refractivity or exhibit a response more similar to the response of a homozygous wild-type GPER patient or cancer. Patients or cancers that are heterozygous may be determined suitable or unsuitable for treatment with LNS8801 depending on other factors, e.g., robustness of prolactin response to a GPER agonist. In some embodiments, the GPER mutant comprises a P16L mutation.

One aspect of the disclosure provides a method for identifying a patient whose cancer can respond to treatment with LNS8801, including obtaining first biological sample(s) from the patient before administering LNS8801; administering an amount of LNS8801 effective to produce a measurable increase in prolactin in a patient, obtaining second biological sample(s) from the patient after administering the LNS8801; analyzing the second sample(s) for an increase in prolactin of greater than about 20% a, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90° %, greater than about 95%, greater than about 100% over the first biological sample(s) after administration of the LNS8801; identifying the patient as one whose cancer can respond to treatment with LNS8801 if a greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 100% increase in prolactin is measured.

One aspect of the disclosure provides a method for selecting a cancer patient suitable for treatment with LNS8801, including obtaining first biological sample(s) from the patient before administering LNS8801; administering an amount of LNS8801 effective to produce a measurable increase in prolactin in a patient; obtaining second biological sample(s) from the patient after administering the LNS8801; analyzing the sample(s) for an increase in prolactin of greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 100% over the first sample(s) after administration of the LNS8801; selecting the patient for treatment with LNS8801 if a greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 100% increase in prolactin is measured.

In some embodiments, the prolactin increase is calculated by dividing the average serum prolactin concentration at about 4, about 7 and about 10 hours after LNS8801 administration by the average prolactin concentration pre-dose, and about 0.5, about 1 and about 2 hours after administration.

One aspect of the disclosure provides a method of treating cancer in a patient in need thereof, including obtaining a biological sample from the patient; determining if the patient is heterozygous or homozygous for wildtype GPER from the sample; determining the patient is amenable to treatment with LNS8801 if heterozygous or homozygous for wildtype GPER; and administering to the patient an effective amount of LNS8801.

One aspect of the disclosure provides a method of treating cancer in a patient in need thereof, including obtaining first biological sample(s) from the patient before administering a GPER agonist; administering an amount of G protein-coupled estrogen receptor 1 (GPER) agonist effective to produce a measurable change in one or more biomarkers of GPER activity in a patient heterozygous or homozygous for wildtype GPER; obtaining second biological sample(s) from the patient at one or more times after administering the GPER agonist; analyzing the sample(s) for a change in the biomarker(s) after administration of the GPER agonist; determining the patient is amenable to treatment with LNS8801 if a measurable change in one or more biomarkers of GPER activity is measured; and administering to the patient an effective amount of LNS8801.

Methods of treating or preventing a disease or disorder in a subject in need thereof comprise administering to a subject a therapeutically effective amount of a LNS8801 where treatment with LNS8801 is acting as an adjuvant prior to, with, or after one or more additional therapies selected from surgical therapy, chemotherapy, anti-PD-1 therapy, targeted molecular or anti-proliferative therapy or radiofrequency ablation therapy.

Another aspect of the disclosure provides methods of treating or preventing cancer, preventing the reoccurrence of cancer, inhibiting the progression of cancer, shrinking a cancer prior to additional therapy, or reducing circulating tumor cells or metastases prior to additional therapy in a subject in need thereof comprising administering to a subject a therapeutically effective amount of cancer drug, GPER agonist, or LNS8801, according to any embodiment disclosed herein.

In some embodiments of the various aspects of the disclosure, the cancer is selected from the group consisting of reproductive cancers, hormone-dependent cancers, leukemia, colorectal cancer, prostate cancer, breast cancer, ovarian carcinoma, endometrial cancer, uterine carcinosarcoma, stomach cancer, rectal cancer, liver cancer, pancreatic cancer, lung cancer, uterine cancer, cervical cancer, cervix uteri cancer, corpus uteri cancer, ovary cancer, testicular cancer, bladder cancer, renal cancer, brain/CNS cancer, head and neck cancer, throat cancer, Hodgkin's disease, non-Hodgkin's lymphoma, multiple myeloma, melanoma, acute leukemia, lymphocytic leukemia, hairy cell leukemia, acute myelogenous leukemia, Ewing's sarcoma, small cell lung cancer, non-small cell lung cancer, choriocarcinoma, rhabdomyosarcoma, Wilms's Tumor, neuroblastoma, cancer of the mouth/pharynx, cancer of the esophagus, cancer of the larynx, kidney cancer, lymphoma, Burkitt lymphoma, sarcoma, angiosarcoma, glioblastoma, medulloblastoma, astrocytoma, and Merkel cell carcinoma.

In particular embodiment, the cancer is selected from the group consisting of melanoma, colorectal cancer, non-small cell lung cancer, and pancreatic cancer.

In some embodiments, the methods may include the co-administration (concurrent, coincident or sequential administration) of one or more additional therapeutic agents. In embodiments, co-administration may be part of the same pharmaceutical composition comprising an enantiomerically purified LNS8801 (SRR G-1), or a derivative thereof, or separate pharmaceutical compositions comprising an enantiomerically purified LNS8801, or a derivative thereof, described herein. In embodiments, co-administration may be at the same time, substantially the same time, before or after administration of the compositions described herein.

The additional therapeutic agents may be selected from the group consisting of an immunotherapy agent (e.g., immune checkpoint therapy agent), a chemotherapy agent, a targeted kinase inhibitor, a histone deacetylase inhibitor, an anti-infective agent, a bromodomain inhibitor, and combinations thereof.

The immunotherapy agent may be selected from the group consisting of PD-1 inhibitors (pembrolizurmab, nivolumab, cemiplimab, JTX-4014, spartalizumab, camrelizumab, sintilimab, tislelizumab, toripalimab, dostarlimab, INCMGA00012 (MGA012), AMP-224, anti-PD-1 and AMP-514), PD-L inhibitors (i.e. Atezolizumab, Avelumab, Durvalumab, anti-PD-LI), CTLA-4 inhibitors (i.e. Ipilimumab, anti-87-1/B7-2, anti-CTLA-4), IL-2, IL-7, IL-12, Oncolytic Viruses (Talimogene Laherparepvec), cytosine phosphate-guanosine, oligodeoxynucleotides, Imiquimod, Resiquimod, and antibodies targeting T cell immunoreceptor with Ig and ITIM domains (TIGIT), inducible co-stimulator (ICOS), Lymphocyte activation gene 3 (LAG-3), I-cell immunoglobulin and Mucin domain containing molecule 3 (TIM3), V-domain containing IG supressor of T cell activation (VISTA), OX40, Glucocorticoid-induced TNF receptor (GITR). CD40, CD47, CD94/NKG2A, Killer immunoglobulin receptor (KIR), and combinations thereof.

The chemotherapy agent may be selected from the group consisting of Cyclophosphamide, methotrexate, 5-fluorouracil, Doxorubicin, Docetaxel, bleomycin, vinblastine, dacarbazine, Mustine, vincristine, procarbazine, etoposide, cisplatin, Epirubicin, capecitabine, folinic acid, oxaliplatin, temozolomide, taxanes, and combinations thereof.

The targeted kinase inhibitor may be selected from the group consisting of Vemurafenib, Dabrafenib, Trametinib, Vandetanib, SU6656, Sunitinib, Sorafenib, Selumetinib, Ruxolitinib, Pegaptanib, Pazopanib, Nilotinib, Mubritinib, Lenvatinib, Lapatinib, Imatinib, Ibrutinib, Gefitinib, Fostamatinib, Erlotinib, Erdafitinib, Dasatinib, Cabozantinib, Crizotinib, Cobimetinib, Cetuximab, Bosutinib, Binimetinib, Axitinib, Afatinib, Adavosertib, and combinations thereof.

The histone deacetylase inhibitor may be selected from the group consisting of Vorinostat, Romidepsin, Chidamide, Panobinostat, Belinostat, Valproic acid, Givinostat, and combinations thereof.

The anti-infective agent may be selected from the group consisting of oritavancin (Orbactiv), dalvavancin (Dalvance), tedizolid phosphate, (Sivextro), clindamycin, linezolid (Zyvox), mupirocin (Bactroban), trimethoprim, sulfamethoxazole, trimethoprim-sulfamethoxazole (Septra or Bactrim), a tetracycline, vancomycin, daptomycin, fluoroquinolines, and combinations thereof.

The bromodomain inhibitor may be selected from the group consisting of OTX015/MK-8628, CPI-0610, BMS-986158, ZEN003694, GSK2820151, GSK525762, INCB054329, INCB057643, ODM-207, R06870810, BAY1238097, CC-90010, AZD5153, FT-1101, ABBV-744, RVX-000222, and combinations thereof.

In some embodiments of the various aspects herein, the GPER agonist includes 2-methoxyestradiol, aldosterone, estradiol, ethynylestradiol, LNS8801, G-1, genistein, hydroxytyrosol, niacin, nicotinamide, quercetin, and resveratrol, and in some embodiments the GPER agonist is LNS8801.

In some embodiments of the various aspects of the disclosure, the patient is homozygous for wildtype GPER, and in some embodiments, the patient is homozygous for a GPER mutant. Patients homozygous for wild-type GPER respond to GPER agonists by upregulation of their native activity. Genetic mutants, once transcribed and translated, typically reduce or eliminate native function. However, in some embodiments, a GPER mutant may result in upregulation of “native” GPER activity upon binding a GPER agonist. In some embodiments of the various aspects of the disclosure, the patient is heterozygous for wildtype GPER, i.e., having one wild-type GPER allele and one mutant GPER allele.

Homozygosity for wild-type or mutant GPER and heterozygosity for GPER (allelic status) can be determined by genotyping according to any method known in the art, e.g., by restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, hybridization to DNA microarrays or beads.

In some embodiments of the various aspects of the disclosure, the test compound, the cancer drug, the GPER agonist, and the LNS8801 are administered in one dose or in two or more doses. One of skill in the art can determine pharmacokinetic and pharmacodynamic characteristics of a particular test compound, cancer drug, GPER agonist, or LNS8801, that determine whether more than one dose is preferable to a single dose.

In embodiments of the various aspects of the disclosure, where the effective amount of test compound, GPER agonist, LNS8801 or cancer drug can be a clinical dose (therapeutically effective amount), a sub-clinical dose, or a microdose, the clinical dose may be about 0.01 mg to about 1000 mg, about 0.01 mg to about 900 mg, about 0.01 mg to about 800 mg, about 0.01 mg to about 700 mg, about 0.01 mg to about 600 mg, about 0.01 mg to about 500 mg, about 0.01 mg to about 400 mg, about 0.01 mg to about 300 mg, about 0.01 mg to about 200 mg, about 0.01 mg to about 100 mg, 0.1 mg to about 1000 mg, about 0.1 mg to about 900 mg, about 0.1 mg to about 800 mg, about 0.1 mg to about 700 mg, about 0.1 mg to about 600 mg, about 0.1 mg to about 500 mg, about 0.1 mg to about 400 mg, about 0.1 mg to about 300 mg, about 0.1 mg to about 200 mg, about 0.1 mg to about 100 mg, about 1 mg to about 1000 mg, about 1 mg to about 900 mg, about 1 mg to about 800 mg, about 1 mg to about 700 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 10 mg to about 1000 mg, about 50 mg to about 1000 mg, about 100 mg to about 1000 mg, about 200 mg to about 1000 mg, about 300 mg to about 1000 mg, about 400 mg to about 1000 mg, about 500 mg to about 1000 mg, about 10 mg to about 500 mg, about 50 mg to about 500 mg, about 100 mg to about 500 mg, about 10 mg to about 300 mg, about 50 mg to about 300 mg, from about 100 mg to about 300 mg, about 10 mg to about 150 mg, about 50 mg to about 150 mg, about 60 mg to about 120 mg, about 50 mg to about 120 mg or a range between any two of these values. Specific examples include, for example, about 1000 mg, about 900 mg, about 800 mg, about 700 mg, about 750 mg, about 600 mg, about 500 mg, about 400 mg, about 450 mg, about 300 mg, about 250 mg, about 200 mg, about 175 mg, about 150 mg, about 125 mg, about 120 mg, about 110 mg, about 100 mg, about 90 mg, about 80 mg, about 70 mg, about 60 mg, about 50 mg, about 30 mg, about 20 mg, about 10 mg, about 5 mg, about 1 mg, about 0.1 mg, about 0.01 mg, or any value between the ranges disclosed above. In some embodiments, the sub-clinical dose includes between about 1.1% and 99.9 percent of the clinical dose, and in some embodiments, the microdose comprises between about 0.01% and 1% of the clinical dose.

In some embodiments, a clinical dose can vary according to, for example, the particular use for which the treatment is made, the manner of administration of the compound or composition, the health and condition of the patient, and the judgment of the prescribing physician. The proportion or concentration of a compound or composition in a pharmaceutical composition comprising, e.g., a GPER agonist such as LNS8801 can vary depending upon a number of factors including chemical characteristics (e.g., hydrophobicity), and the route of administration. For example, the compounds or compositions can be provided in an aqueous physiological buffer solution containing about 0.1 to about 10% w/v of the compound or composition for parenteral administration. Some typical dose ranges for the compounds or compositions are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound or composition selected, formulation of the excipient, and its route of administration. Effective doses can be extrapolated from dose-response curves derived from in vitro or animal model test systems.

In some embodiments of the various aspects of the disclosure, the biological sample(s) include one or more cells and/or tissues that are not cancerous, a negligible number (e.g., <5%, <2%, <1%) of cancerous cells (or cell products, and the like), and in some embodiments, the biological sample(s) include cells and/or tissues that are all non-cancerous.

In some embodiments, the first biological sample(s) are obtained not more than 30 days prior to administering the test compound, cancer drug, the GPER agonist, or the LNS8801, and in some embodiments, the first biological sample(s) is collected at the same time of day as the second biological sample(s). In some embodiments, the first biological sample(s) are obtained immediately before administration of the test compound, the cancer drug, the GPER agonist, or the LNS8801. In some embodiments, the biomarker(s) of the test compound, the cancer drug, the GPER agonist, and the LNS8801 activity include one or more molecular biomarkers, imaging biomarkers or non-invasively measurable biomarkers, which biomarkers include, in embodiments, circulating biomarker(s) and/or systemic biomarker(s), and/or biomarker(s), as described elsewhere herein, that are localized to the first and/or second biological sample(s).

In some embodiments that include administering an effective amount of GPER agonist or LNS8801, the biomarker(s) comprise circulating biomarker(s), which include a change in prolactin level, insulin level, c-Myc and/or glucose level, which change, in embodiments, includes an increase in prolactin level or activity, an increase in insulin level or activity or a decrease in c-Myc level or activity. In some embodiments, the biomarker of GPER agonist activity (including LNS88801) is an increase in circulating prolactin level.

In some embodiments wherein the biomarker of GPER agonist activity (including LNS88801) is an increase in circulating prolactin level, the prolactin exhibits an about 1.25-fold induction, an about 1.30-fold induction, an about 1.35-fold induction, an about 1.40-fold induction, an about 1.45-fold induction, an about 1.50-fold induction, an about 1.55-fold induction, an about 1.60-fold induction, an about 1.65-fold induction, an about 1.70-fold induction, an about 1.75-fold induction, an about 1.80-fold induction, an about 1.85-fold induction, an about 1.90-fold induction, an about 1.95-fold induction, an about 2.0-fold induction after administration of an effective amount of GPER agonist, including LNS8801.

In some embodiments wherein the biomarker of GPER agonist activity (including LNS88801) is an increase in circulating prolactin level, the prolactin increases above a threshold at an average of about 4 hours (+/−20 min), about 7 hours (+/−45 min) and about 12 hours (+/−2 hours), or, in some embodiments about 10 hours, divided by the average concentration of prolactin at pre-dose and 30 min, 1 hour and 2 hours post-dose, and the increase is more than 25% to monotherapy or more than 40% to monotherapy and less to combination therapy with a PD-1 inhibitor.

The following are provided for exemplification purposes only and are not intended to limit the scope of the invention described in broad terms above. All references cited in this disclosure are incorporated herein by reference.

### EXAMPLES

### Example 1: LNS8801 Pharmacokinetics

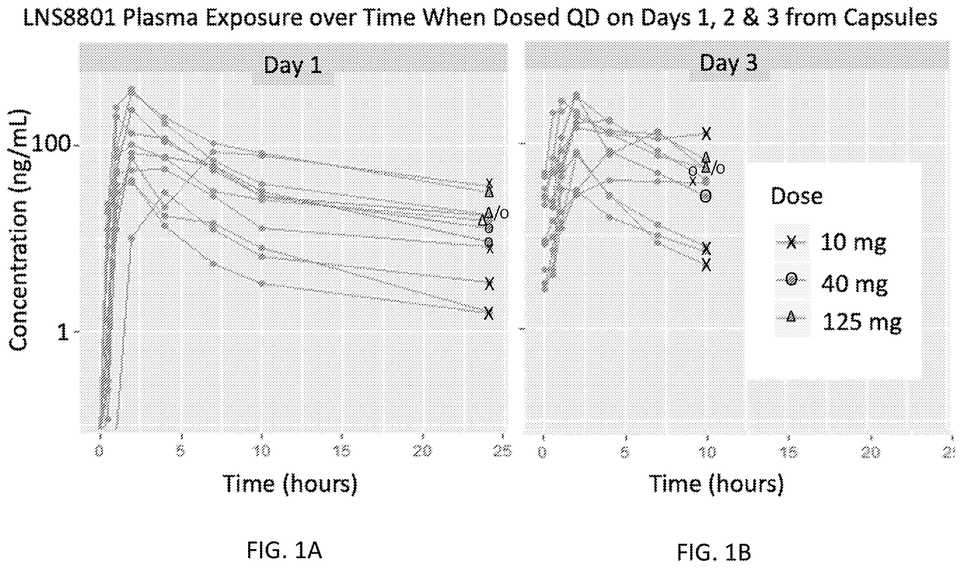

Patients with advanced cancer were dosed with 10, 40, and 125 mg of LNS8801 in a capsule on three consecutive days/week. Blood was collected from patients pre-dose and at 0.5, 1, 2, 4, 7, 10, and 24 hours after dosing. Samples were analyzed by mass spectrometry for the concentration of LNS8801. FIG. 1(A) shows the pharmacokinetics of LNS8801 on day 1 of dosing, and FIG. 1(B) shows the phannacokinetics of LNS8801 on day 3 of dosing. LNS8801 is well absorbed, producing plasma exposures that are predicted to be efficacious, even at the lowest dose evaluated. Half-life in humans is approximately 10 hours.

### Example 2: LNS8801-Induced c-Myc Depletion

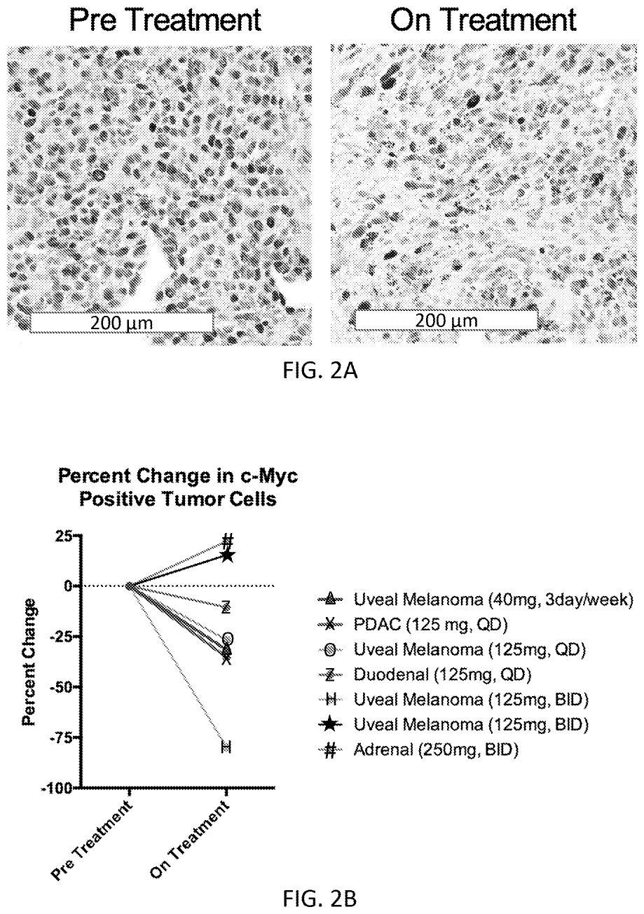

Pre-treatment biopsies were collected within 28 days of initiating treatment, and on-treatment biopsies were collected 8-19 days after LNS8801 treatment began. Tumor samples were formalin-fixed, paraffin-embedded, sectioned, and assessed for c-Myc positive tumor cells by immunohistochemistry by a blinded pathologist. FIG. 2(A) shows representative images of pre and on-treatment biopsies stained for c-Myc (showing a significant decrease in c-Myc on-treatment; uveal melanoma is shown in this example), and FIG. 2(B) shows a quantification of the precent change in c-Myc positive tumor cells after treatment.

In the biopsies tested to date, we have observed a dramatic depletion in c-Myc positive tumor cells after administration of LNS8801 on most samples. Samples without c-Myc depletion either did not have any GPER protein expression or were negative for prolactin, a systemic marker of GPER activity the induction of which requires GPER signaling. LNS8801 treatment induces prolactin in patients that have demonstrated stable disease or stable target lesions, supporting the use of prolactin as a prognostic biomarker.

### Example 3: Prolactin Induction Versus Best RECIST Response after LNS8801 Monotherapy

Blood was collected from patients pre-dose and at 0.5, 1, 2, 4, 7, and 10 hours after dosing and analyzed for the concentration of prolactin. Prolactin induction was calculated by dividing the average of the 4 hour through 10 hour timepoints by the average of pre-dose to 2 hour timepoints. Prolactin-induction was plotted with the best RECIST response of target lesions, and linear regression was performed.

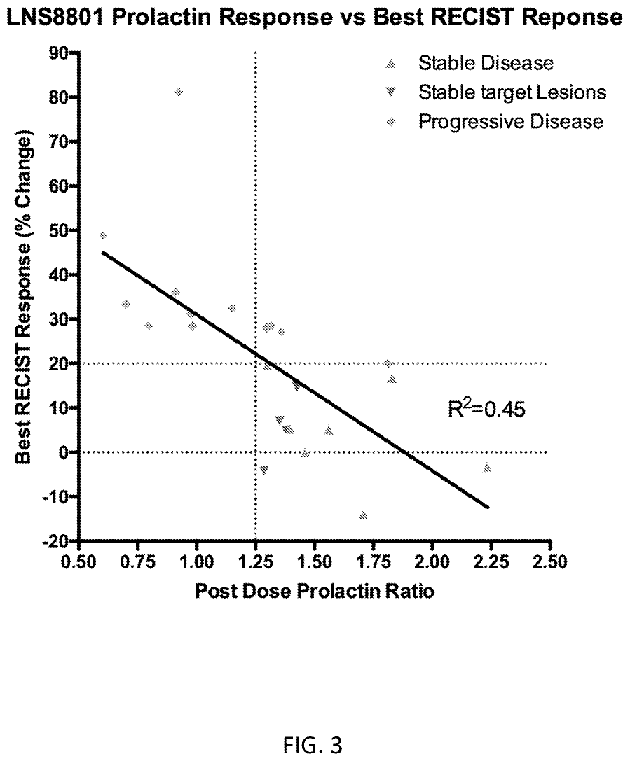

Patients with progressive disease under RECIST typically exhibited a reduction or no change in prolactin levels on treatment (FIG. 3, upper left sextant). In contrast, patients exhibiting stable disease or stable target lesions typically exhibited a substantial induction of prolactin (at least 1.25-fold) on treatment (FIG. 3, center right and lower right sextants). Further, patients exhibiting stable disease (i.e., the most favorable prognosis) typically demonstrated stronger prolactin induction than patients who exhibited stable target lesions (i.e., a less favorable prognosis).

### Example 4: RECIST Response in Prolactin Responders Versus Non-Responders

Blood was collected from patients pre-dose and 0.5, 1, 2, 4, 7, and 10 hours after dosing and analyzed for the concentration of prolactin (PRLX). Prolactin induction was calculated by dividing the average of the 4 hour through 10 hour timepoints by the average of pre-dose to 2 hour timepoints. Best RECIST response of target lesions was plotted in patients without a prolactin response (<1.25-fold PRLX induction) and with a prolactin response (>1.25-fold PRLX induction), and statistics performed using the Mann-Whitney test.

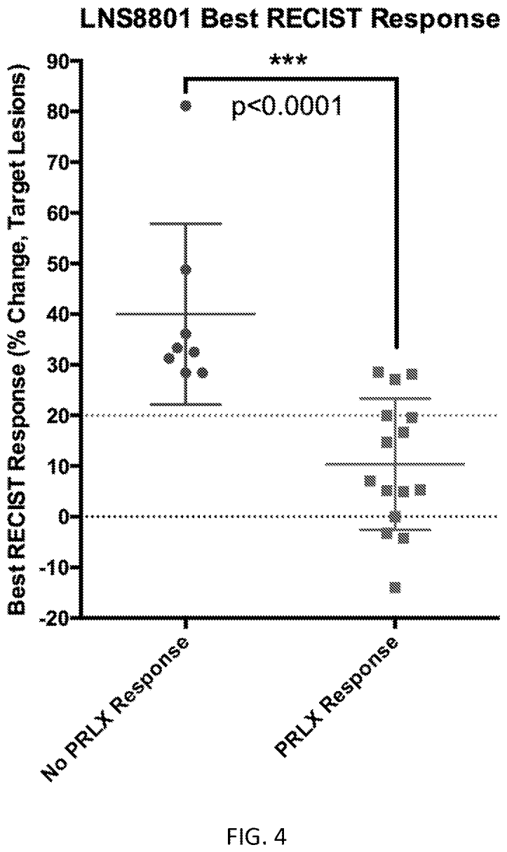

Normally, prolactin levels fall or are stable throughout the day due to circadian rhythm. Consequently, it is notable that all patients that exhibited disease stabilization demonstrated ≥1.25-fold induction; whereas no patient without induction demonstrated disease stabilization. Stratifying the data based upon prolactin-responders vs non-prolactin-responders reveals a highly significant difference in the best RECIST response after LNS8801 treatment (FIG. 4). In evaluable patients, we observed a lack of prolactin response in 35% of the patients, and every patient lacking a prolactin response had progressive disease on LNS8801, indicating the lack of a functional GPER pathway.

Having described the invention in detail and by reference to specific aspects and/or embodiments thereof, it will be apparent that modifications and variations are possible without departing from the scope of the invention defined in the appended claims. Additional aspects and embodiments of the disclosure are provided by the claims below, which can be combined in any number and in any combination not technically or logically inconsistent. Although some aspects of the present invention may be identified herein as particularly advantageous, it is contemplated that the present invention is not limited to these particular aspects of the invention.

What is claimed is:

1. A method for identifying a patient whose cancer can respond to treatment with a cancer drug that binds to a cancer target in a target pathway, comprising:
obtaining first non-cancerous biological sample(s) from the patient at one or more times before administering a test compound;
administering an amount of test compound effective to produce a measurable change in one or more biomarkers in the target pathway;
obtaining second non-cancerous biological sample(s) from the patient at one or more times after administering the test compound;
analyzing the second non-cancerous biological sample(s) for a change in the biomarker(s) after administration of the test compound as compared to the first sample(s); and
identifying the patient as one whose cancer can respond to treatment with the cancer drug if the measurable change in one or more biomarkers in the target pathway corresponds to the measurable change in a healthy subject.

2. A method for identifying a cancer patient suitable for treatment with a cancer drug that binds to a cancer target in a target pathway, comprising:
obtaining first non-cancerous biological sample(s) from the patient at one or more times before administering a test compound;
administering an amount of test compound effective to produce a measurable change in one or more biomarkers in the target pathway;
obtaining second non-cancerous biological sample(s) from the patient at one or more times after administering the test compound;
analyzing the second non-cancerous biological sample(s) for a change in the biomarker(s) after administration of the test compound as compared to the first sample(s); and
identifying the cancer patient as suitable for treatment with the cancer drug if the measurable change in one or more biomarkers in the target pathway is substantially similar to the measurable change in a one or more cancer patients who responded to the cancer drug.

3. The method of either one of claims 1-2, wherein the test compound comprises an agonist of the cancer target.

4. The method of either one of claims 1-2, wherein the test compound comprises an antagonist of the cancer target.

5. The method of either one of claims 1-2, wherein the test compound comprises the cancer drug.

6. The method of either one of claims 1-2, wherein the effective amount of test compound is administered in one dose.

7. The method of either one of claims 1-2, wherein the effective amount of test compound is administered in two or more doses.

8. The method of either one of claims 1-2, wherein the effective amount of test compound is selected from a clinical dose, a sub-clinical dose, or a microdose.

9. The method of claim 8, wherein the sub-clinical dose comprises between about 1.1% and 99.9 percent of the clinical dose.

10. The method of claim 8, wherein the microdose comprises between about 0.01% and 1% of the clinical dose.

11. The method of either one of claims 1-2, wherein the first biological sample(s) are obtained not more than 30 days prior to administering the test compound.

12. The method of either one of claims 1-2, wherein the first biological sample(s) is collected at the same time of day as the second biological sample(s).

13. The method of either one of claims 1-2, wherein the biomarker(s) of test compound activity comprise one or more molecular biomarkers, imaging biomarkers or non-invasively measurable biomarkers.

14. The method of claim 13, wherein the molecular biomarker(s) comprise circulating biomarker(s).

15. The method of any one of claims 1-14, wherein at least one biomarker is the cancer target of the cancer drug.

16. The method of any one of claims 1-14, wherein the one or more biomarker(s) is not the cancer target of the cancer drug.

17. The method of any one of claims 1-16, wherein the biomarker(s) are systemic biomarkers and/or circulating biomarkers.

18. The method of any one of claims 1-16, wherein the biomarker(s) are localized to the first and/or second biological sample(s).

19. A method for identifying a patient whose cancer can respond to treatment with LNS8801, comprising:
obtaining first biological sample(s) from the patient at one or more times before administering a G protein-coupled estrogen receptor 1 (GPER) agonist;
administering an amount of GPER agonist effective to produce a measurable change in one or more biomarkers of GPER activity in the patient;
obtaining second biological sample(s) from the patient at one or more times after administering the GPER agonist;
analyzing the second sample(s) for a change in the biomarker(s) after administration of the GPER agonist as compared to the first sample(s);
identifying the patient as one whose cancer can respond to treatment with LNS8801 if a measurable change in one or more biomarkers of GPER activity is measured.

20. A method for identifying a patient whose cancer can respond to treatment with LNS8801, comprising:
obtaining first biological sample(s) from the patient at one or more times before administering a G protein-coupled estrogen receptor 1 (GPER) agonist;
administering an amount of GPER agonist effective to produce a measurable change in one or more biomarkers of GPER activity in a patient heterozygous or homozygous for wildtype GPER;
obtaining second biological sample(s) from the patient at one or more times after administering the GPER agonist;
analyzing the second sample(s) for a change in the biomarker(s) after administration of the GPER agonist as compared to the first sample(s);
identifying the patient as one whose cancer can respond to treatment with LNS8801 if a measurable change in one or more biomarkers of GPER activity is measured.

21. A method for selecting a cancer patient suitable for treatment with LNS8801, comprising:
obtaining first biological sample(s) from the patient at one or more times before administering a GPER agonist;
administering an amount of GPER agonist effective to produce a measurable change in one or more biomarkers of GPER activity in a patient;
obtaining second biological sample(s) from the patient at one or more times after administering the GPER agonist;
analyzing the second sample(s) for a change in the biomarker(s) after administration of the GPER agonist as compared to the first sample(s);
selecting the patient for treatment with LNS8801 if a measurable change in one or more biomarkers of GPER activity is measured.

22. A method for identifying a patient whose cancer can respond to treatment with LNS8801, comprising:
obtaining a biological sample;
analyzing the sample to determine if the patient is heterozygous or homozygous for wildtype GPER;
identifying the patient as one whose cancer can respond to treatment with LNS8801 if heterozygous or homozygous for GPER.

23. A method for selecting a cancer patient suitable for treatment with LNS8801, comprising:
obtaining a biological sample;
analyzing the sample to determine if the patient is heterozygous or homozygous for wildtype GPER;
selecting the patient for treatment with LNS8801 if heterozygous or homozygous for GPER.

24. A method for identifying a patient whose cancer will be refractive to treatment with LNS8801, comprising:
obtaining a biological sample;
analyzing the sample to determine if GPER is localized in the nucleus;
identifying the patient as one whose cancer will be refractive to treatment with LNS8801 if GPER is localized in the nucleus.

25. A method for identifying a patient whose cancer can be refractive to treatment with LNS8801, comprising:
obtaining a biological sample;
analyzing the sample to determine if the patient is heterozygous or homozygous for a GPER mutant;
identifying the patient as one whose cancer can be refractive to treatment with LNS8801 if heterozygous or homozygous for a GPER mutant.

26. A method for selecting a cancer patient unsuitable for treatment with LNS8801, comprising:
obtaining a biological sample;
analyzing the sample to determine if the patient is heterozygous or homozygous for a GPER mutant;
selecting the patient as unsuitable for treatment with LNS8801 if heterozygous or homozygous for a GPER mutant.

27. The method of either one of claims 25-26, wherein the GPER mutant comprises a P16L mutation.

28. A method for identifying a patient whose cancer can respond to treatment with LNS8801, comprising:
obtaining first biological sample(s) from the patient at one or more times before administering LNS8801;
administering an amount of LNS8801 effective to produce a measurable increase in prolactin in a patient;
obtaining second biological sample(s) from the patient at one or more times after administering the LNS8801;
analyzing the second sample(s) for an increase in prolactin of greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, greater than about 100% over the first biological sample(s) after administration of the LNS8801;
identifying the patient as one whose cancer can respond to treatment with LNS8801 if a greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 100% increase in prolactin is measured.

29. A method for selecting a cancer patient suitable for treatment with LNS8801, comprising:
obtaining first biological sample(s) from the patient at one or more times before administering LNS8801;
administering an amount of LNS8801 effective to produce a measurable increase in prolactin in a patient;
obtaining second biological sample(s) from the patient at one or more times after administering the LNS8801;
analyzing the sample(s) for an increase in prolactin of greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 100% over the first sample(s) after administration of the LNS8801;
selecting the patient for treatment with LNS8801 if a greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or greater than about 100% increase in prolactin is measured.

30. The method of either one of claims 28-29, wherein the increase in prolactin in the second sample(s) is greater than about 25% over the first biological sample(s).

31. The method of either one of claims 28-29, wherein the increase in prolactin in the second sample(s) is greater than about 30% over the first biological sample(s).

32. The method of either one of claims 28-29, wherein the increase in prolactin in the second sample(s) is greater than about 35% over the first biological sample(s).

33. The method of either one of claims 28-29, wherein the increase in prolactin in the second sample(s) is greater than about 40% over the first biological sample(s).

34. The method of either one of claims 28-29, wherein the increase in prolactin in the second sample(s) is greater than about 45% over the first biological sample(s).

35. The method of either one of claims 28-29, wherein the increase in prolactin in the second sample(s) is greater than about 50% over the first biological sample(s).

36. The method of either one of claims 28-29, wherein the increase in prolactin in the second sample(s) is greater than about 55% over the first biological sample(s).

37. The method of either one of claims 28-29, wherein the increase in prolactin in the second sample(s) is greater than about 60% over the first biological sample(s).

38. The method of either one of claims 28-29, wherein the increase in prolactin in the second sample(s) is greater than about 65% over the first biological sample(s).

39. The method of either one of claims 28-29, wherein the increase in prolactin in the second sample(s) is greater than about 70% over the first biological sample(s).

40. The method of either one of claims 28-29, wherein the increase in prolactin in the second sample(s) is greater than about 75% over the first biological sample(s).

41. The method of either one of claims 28-29, wherein the increase in prolactin in the second sample(s) is greater than about 80% over the first biological sample(s).

42. The method of either one of claims 28-29, wherein the increase in prolactin in the second sample(s) is greater than about 85% over the first biological sample(s).

43. The method of either one of claims 28-29, wherein the increase in prolactin in the second sample(s) is greater than about 90% over the first biological sample(s).

44. The method of either one of claims 28-29, wherein the increase in prolactin in the second sample(s) is greater than about 95% over the first biological sample(s).

45. The method of either one of claims 28-29, wherein the increase in prolactin in the second sample(s) is greater than about 100% over the first biological sample(s).

46. The method of any one of claims 19-23 or 28-45, wherein the patient is homozygous for wildtype GPER.

47. The method of any one of claims 25-26, wherein the patient is homozygous for a GPER mutant.

48. The method of any one of claims 28-47, wherein the prolactin increase is calculated by dividing the average serum prolactin concentration at about 4, about 7 and about 10 hours after LNS8801 administration by the average prolactin concentration pre-dose, and about 0.5, about 1 and about 2 hours after administration.

49. A method of treating cancer in a patient in need thereof, comprising:
obtaining a biological sample from the patient;
determining if the patient is heterozygous or homozygous for wildtype GPER from the sample;
determining the patient is amenable to treatment with LNS8801 if heterozygous or homozygous for wildtype GPER; and
administering to the patient an effective amount of LNS8801.

50. A method of treating cancer in a patient in need thereof, comprising:
obtaining first biological sample(s) from the patient at one or more times before administering a GPER agonist;
administering an amount of G protein-coupled estrogen receptor 1 (GPER) agonist effective to produce a measurable change in one or more biomarkers of GPER activity in a patient heterozygous or homozygous for wildtype GPER;
obtaining second biological sample(s) from the patient at one or more times after administering the GPER agonist;
analyzing the sample(s) for a change in the biomarker(s) after administration of the GPER agonist;
determining the patient is amenable to treatment with LNS8801 if a measurable change in one or more biomarkers of GPER activity is measured; and
administering to the patient an effective amount of LNS8801.

51. The method of any one of claims 49-50, wherein the patient is homozygous for wildtype GPER.

52. The method of any one of claims 49-51, wherein the effective amount of GPER agonist is administered in one dose.

53. The method of any one of claims 49-51, wherein the effective amount of GPER agonist is administered in two or more doses.

54. The method of any one of claims 49-51, wherein the effective amount of GPER agonist is selected from a clinical dose, a sub-clinical dose or a microdose.

55. The method of claim 54, wherein the sub-clinical dose comprises between about 1.1% and 99.9 percent of the clinical dose.

56. The method of claim 54, wherein the microdose comprises between about 0.01% and 1% of the clinical dose.

57. The method of any one of claims 49-56, wherein the GPER agonist comprises 2-methoxyestradiol, aldosterone, estradiol, ethynylestradiol, LNS8801, G-1, genistein, hydroxytyrosol, niacin, nicotinamide, quercetin, and resveratrol.

58. The method of claim 57, wherein the GPER agonist is LNS8801.

59. The method of any one of claims 49-51, wherein the first biological sample(s) are obtained not more than 30 days prior to administering the GPER agonist.

60. The method of any one of claims 49-51, wherein the first biological sample(s) is collected at the same time of day as the second biological sample(s).

61. The method of any one of claims 49-51, wherein the biomarker(s) of GPER activity comprise one or more molecular biomarkers, imaging biomarkers or non-invasively measurable biomarkers.

62. The method of claim 61, wherein the molecular biomarker(s) comprise circulating biomarker(s).

63. The method of either one of claims 61-62, wherein the molecular biomarker(s) comprise a change in prolactin level, insulin level, c-Myc and/or glucose level.

64. The method of any one of claims 61-63, wherein the molecular biomarker(s) comprise an increase in prolactin level or activity, an increase in insulin level or activity or a decrease in c-Myc level or activity.

65. The method of any one of claims 61-64, wherein the biomarker of GPER activity is an increase in circulating prolactin level.

66. The method of any one of claims 61-65, wherein the prolactin exhibits an about 1.25-fold induction after administration of an effective amount of GPER agonist.

67. The method of any one of claims 61-65, wherein the prolactin exhibits an about 1.3-fold induction after administration of an effective amount of GPER agonist.

68. The method of any one of claims 61-65, wherein the prolactin exhibits an about 1.35-fold induction after administration of an effective amount of GPER agonist.

69. The method of any one of claims 61-65, wherein the prolactin exhibits an about 1.4-fold induction after administration of an effective amount of GPER agonist.

70. The method of any one of claims 61-65, wherein the prolactin exhibits an about 1.45-fold induction after administration of an effective amount of GPER agonist.

71. The method of any one of claims 61-65, wherein the prolactin exhibits an about 1.5-fold induction after administration of an effective amount of GPER agonist.

72. The method of any one of claims 61-65, wherein the prolactin exhibits an about 1.55-fold induction after administration of an effective amount of GPER agonist.

73. The method of any one of claims 61-65, wherein the prolactin exhibits an about 1.6-fold induction after administration of an effective amount of GPER agonist.

74. The method of any one of claims 61-65, wherein the prolactin exhibits an about 1.65-fold induction after administration of an effective amount of GPER agonist.

75. The method of any one of claims 61-65, wherein the prolactin exhibits an about 1.7-fold induction after administration of an effective amount of GPER agonist.

76. The method of any one of claims 61-65, wherein the prolactin exhibits an about 1.75-fold induction after administration of an effective amount of GPER agonist.

77. The method of any one of claims 61-65, wherein the prolactin exhibits an about 1.8-fold induction after administration of an effective amount of GPER agonist.

78. The method of any one of claims 61-65, wherein the prolactin exhibits an about 1.85-fold induction after administration of an effective amount of GPER agonist.

79. The method of any one of claims 61-65, wherein the prolactin exhibits an about 1.9-fold induction after administration of an effective amount of GPER agonist.

80. The method of any one of claims 61-65, wherein the prolactin exhibits an about 2.0-fold induction after administration of an effective amount of GPER agonist.

81. The method of any one of claims 61-65, wherein the prolactin increases above a threshold at an average of about 4 hours (+/−20 min), about 7 hours (+1-45 min) and about 12 hours (+/−2 hours) divided by the average concentration of prolactin at pre-dose and 30 min, 1 hour and 2 hours post-dose, and
wherein the increase is more than 25% to monotherapy and less to combination therapy with a PD-1 inhibitor.

82. The method of any one of claims 61-65, wherein the prolactin increases above a threshold at an average of about 4 hours (+/−20 min), about 7 hours (+/−45 min) and about 12 hours (+/−2 hours) divided by the average concentration of prolactin at pre-dose and 30 min, 1 hour and 2 hours post-dose, and
wherein the increase is more than 40% to monotherapy and less to combination therapy with a PD-1 inhibitor.

83. The method of claim 61, wherein the non-invasively measurable biomarker(s) comprises flushing or a change in blood pressure.

84. The method of any one of claims 49-51, further comprising concurrently, coincidently or sequentially administering a PD-1 inhibitor comprising one or more of pembrolizumab, nivolumab, cemiplimab, JTX-4014, spartalizumab, camrelizumab, sintilimab, tislelizumab, toripalimab, dostarlimab, INCMGA00012 (MGA012), AMP-224, and AMP-514.

85. The method of claim 84, wherein the PD-1 inhibitor comprises pembrolizumab.

86. The method of claim 85, wherein the PD-1 inhibitor is pembrolizumab.

87. The method of any one of claims 19-86, wherein the biological sample(s) comprise one or more cells and/or tissues that are not cancerous.
